(19) United States
(12) Patent Application Publication
(10) Pub. No.: US 2026/0283502 A1
(43) Pub. Date: Sep. 24, 2026

(54) ANTENNA ARRANGEMENT FOR ELECTROMAGNETIC WAVES IN NEAR-BODY APPLICATIONS WITH REDUCED FREE SPACE RADIATION

(71) Applicant: ALTAVO GMBH, Dresden (DE)

(72) Inventors: Martin LAABS, Dresden (DE); Dirk PLETTEMEIER, Dresden (DE)

(21) Appl. No.: 18/874,155

(22) PCT Filed: Jun. 14, 2023
(86) PCT No.: PCT/EP2023/066003

§ 371 Date: Dec. 12, 2024

(30) Foreign Application Priority Data
Jun. 15, 2022 (DE) 10 2022 115 031.3

(51) Int. Cl. A61B 5/107; A61B 5/05; G10L 15/25; H01Q 1/27; H01Q 17/00

(52) U.S. Cl. CPC A61B 5/1075; A61B 5/05; G10L 15/25; H01Q 1/273; H01Q 17/00

(57) ABSTRACT

An antenna arrangement in particular for radar measurements, that can be used close to the body, e.g., stuck to the skin. The antenna arrangement has a particularly reduced radiation into free space.

### TECHNICAL FIELD

Various examples of the disclosure relate to an antenna arrangement for electromagnetic waves that is used in a near-body application and by means of which radiation of the electromagnetic waves into the free space facing away from the body can be reduced.

### BACKGROUND

Medical applications for radar measurements are known. They use antenna arrangements in near-body applications to employ electromagnetic waves to measure a person's anatomical structures.

An example relates to the characterization of the vocal tract. The characterization of the vocal tract can be carried out in order to determine an intended speech utterance of the person. See, for example, Wagner, Christoph, et al. “Silent Speech Command Word Recognition Using Stepped Frequency Continuous Wave Radar.” (2021).

Technical descriptions of the human voice often use the so-called source-filter model. The lungs, trachea, and larynx together form the source. The air is compressed in the lungs and flows through the trachea up to the larynx. In the larynx, the vocal folds—colloquially known as vocal cords—form the glottis. The muscles of the larynx keep the vocal folds under tension by exerting force through the arytenoid cartilages.

When speaking with a voice, the pressure in the trachea and the tension of the vocal folds cause them to open and close periodically, thus creating an acoustic vibration, a sound wave. This sound wave is acoustically filtered by the time-variable shape of the vocal tract, consisting of the pharynx, oral cavity and nasal cavity, before it exits through the mouth and nostrils.

The production of speech from corresponding speech utterances consists of the process of phonation, expressed in technical terms it is the stimulation of an acoustic vibration by the vocal folds, and articulation, i.e., the filtering of the sound spectrum by the time-variable shape of the vocal tract. The shape of the vocal tract is determined by the soft palate, which opens or closes the nasal cavity, the tongue, the upper and lower rows of teeth, and the upper and lower lips.

Usually, radar measurements require a relatively high resolution. Therefore, the electromagnetic waves used for radar measurements have a comparatively large bandwidth. For example, frequencies in the range from 0.3 GHz to 15 GHz, i.e., in the high-frequency spectrum, could be used. The upper frequency is limited by the increasing attenuation in the tissue and the lowest frequency is limited by the feasible antenna size.

Transmissions operations in this frequency range are typically regulated. If radiation is permitted, the transmission power is typically restricted by regulation in order to limit interferences between different users.

On the other hand, the highest possible transmission power may be desirable to increase the signal-to-noise ratio of the radar measurement. Typically, the achievable levels of the measured signal are limited by the high attenuation of the electromagnetic waves in the tissue.

### SUMMARY

There is a need for techniques to implement radar measurements in near-body applications. In particular, there is a need for antenna arrangements that reduce unwanted free space radiation.

This task is solved by the features specified in the independent claims. The features of the independent claims define embodiments.

This disclosure describes antenna arrangements for electromagnetic waves in near-body applications which emit electromagnetic waves preferably into the tissue and thereby allow for the lowest possible radiation into the free space (i.e., an environment opposite the tissue). Thus, these antenna arrangements make it possible to couple most of the radio frequency energy into the tissue and to radiate only a comparatively smaller portion of the radio frequency energy into the free space.

An antenna arrangement for electromagnetic waves in a near-body application is disclosed. For example, the antenna arrangement could be used to characterize a vocal tract. The vocal tract can be characterized, for example, to determine an intended speech utterance.

The antenna arrangement has a top side and a bottom side. The antenna arrangement is designed to be placed with its bottom side on a person's skin surface. The antenna arrangement comprises a two-dimensional extended carrier structure, e.g., an elastic or flexible carrier structure. The carrier structure could be a plastic substrate. A glass fiber-reinforced epoxy resin could be used. The carrier structure can be flexible. Therefore, the carrier structure, and thus the antenna arrangement, is capable to adapt to the surface shape of the skin surface.

The antenna arrangement also includes one or more conductor structures. In coordination with the remaining parts of the arrangement, the conductor structures are designed to couple the electromagnetic waves into the tissue. They thus form the antenna.

The one or more conductor structures are attached to the carrier structure.

A high-frequency (RF) current can flow in the one or more conductor structures.

The electromagnetic waves can have a frequency in the range from 0.3 to 15 GHZ.

Furthermore, the antenna arrangement also includes one or more absorber elements. These absorber elements are attached directly or indirectly (via one or more other elements or layers) to the carrier structure and are designed to bring about a radiation pattern of the electromagnetic waves that is asymmetrical with respect to the top side and bottom side.

The change in the radiation pattern caused by the absorber elements can lead to a more attenuated and thus reduced radiation into the half-space facing the top side compared to the radiation into the half-space facing the bottom side. Thus, the absorber elements influence the ratio of the radiated power into the two half-spaces in favor of the half-space facing the person's tissue. This means that the so-called front-to-back ratio can be changed in favor of a radiation into the half-space facing the bottom side. In connection with this asymmetrical radiation pattern, a corresponding complementary asymmetrical detection pattern can also result, if the respective antenna arrangement is used to receive electromagnetic waves.

The use of an asymmetrical radiation pattern can reduce the radiation into the free space. This means that—compared to an arrangement without absorber elements—higher transmission powers can be used because, for example, regulatory restrictions regarding the maximum permissible radiation into the free space can be adhered to even at higher transmission powers: the electromagnetic energy is emitted in the direction of the tissue but not or only to a significantly smaller extent into the free space. This in turn allows the implementation of radar measurements with a higher signal-to-noise ratio so that corresponding applications benefit from a higher signal quality.

The signal-to-noise ratio can be further improved, if an appropriate antenna arrangement with asymmetrical detection pattern is used to detect the electromagnetic waves after their propagation in the tissue. The cause for this is the fact that fewer interference signals are received from the free space. The undesired interference is reduced.

The use of absorber elements can, furthermore, provide a particularly compact implementation of the antenna arrangement, especially in comparison to reflectors that would reflect the electromagnetic waves.

There are different implementation variants for the one or more absorber elements. Such implementation variants, which are described in more detail below, could also be used in combination.

In one implementation variant, the one or more absorber elements provide a resistive loss mechanism for electric currents. This means that by using the corresponding one or more resistors, the current distribution in the one or more metallic conductor structures can be specifically shaped such that the asymmetrical radiation pattern is prioritized. For example, a symmetry breaking in the current distribution with respect to the top and bottom can be achieved or promoted by providing corresponding resistive structures at a suitable location with respect to the one or more conductor structures.

For example, if the one or more conductor structures comprise a flat conductor plane, the one or more resistors could be arranged on a circumference of the flat conductor plane and connected to it. The one or more resistors could be designed as a resistive layer or as housed resistors applied to the surface of the conductor plane.

Such a technology is based on the knowledge that current flow in the edge area of the flat conductor plane is particularly responsible for radiation into the free space. By providing one or more resistors, the high-frequency signals can be absorbed in a targeted manner in the edge area of the flat conductor plane, thus reducing the radiation into the free space.

For example, the one or more resistors could be applied to the flat conductor plane and conductively connected to it and extend beyond its circumference. It is conceivable that the one or more resistors extend perpendicularly to the flat conductor plane. For example, the one or more resistors could be implemented as a resistive layer with a perpendicular extension on an edge of the flat conductor plane.

An exemplary implementation variant for the one or more absorber elements that utilize a resistive loss mechanism is described above. Alternatively or in addition to such a utilization of the resistive loss mechanism, it is also conceivable to utilize a dielectric or magnetic loss mechanism for the electromagnetic near field of the one or more metallic conductor structures. Here, it is conceivable, for example, that the one or more absorber elements comprise one or more two-dimensionally extended absorbent material layers. These layers can be applied, for example, to the metallic conductor structures that form the one or more conductor structures (e.g., separated by an insulating layer). An appropriate absorbent material can be selected, for example, from the following group: carbon (such as graphite), metal (such as nickel, titanium or chromium), and metal particles in a carrier material (e.g., metal particles in silicone or graphite dust in epoxy resin; mixtures are also conceivable). Carbon particles in a carrier material are conceivable. The absorbent material can be made of a material mixture that can contain such materials.

In particular, it would be possible in such an example that the one or more absorbent material layers have a lateral structuring. This means that this lateral structuring can be formed in the plane parallel to the top or bottom side. For example, the thickness of the corresponding one or more absorbent material layers could vary. The one or more absorbent material layers could be interrupted by trenches. This lateral structuring can, in particular, correlate with the lateral structuring of the one or more metallic conductor structures. For example, absorbent material could be provided in spaces between metal conductors of the one or more conductor structures, or directly above the metal conductors, or above edges of the metal conductors. By the use of a lateral structuring, an increased robustness of the antenna arrangement with respect to repeated bending can be achieved.

Such techniques as described above make it particularly possible to provide the antenna arrangement with a thickness (i.e., perpendicular to the two-dimensionally extended carrier structure) that is comparatively small. For example, an antenna arrangement can be implemented the extent of which perpendicular to the carrier structure is not greater than one hundredth of the wavelength of the longest wavelength component of the electromagnetic waves in vacuum. Such a thin design of the antenna arrangement makes it possible to wear this antenna arrangement comfortably on the skin, even during longer periods of time. In particular, the flexible design of the carrier structure ensures that the antenna arrangement keeps its flexibility and elasticity so that the antenna arrangement can adapt to the surface shape of the skin.

As a general rule, there are different ways to implement the carrier structure or the other components of the antenna arrangement. For example, it is conceivable for that the carrier structure is designed as a textile layer. This means that the carrier structure can consist of a woven material or be formed by the textile. Textiles or so-called non-woven materials are conceivable. Knitted or knotted layers would be possible. In such an example, the one or more conductor structures could, for example, be woven or embroidered into the textile layer as threads or ribbons or be fixed in another way. The absorber elements could be attached to these threads or ribbons or connected to them or applied to the textile layer that forms the carrier structure.

In a further example, it is conceivable that the carrier structure and the one or more conductor structures are made of a film, such as polyimide film. In this case, the carrier structure and the one or more conductor structures can be implemented as thin layers with elasticity on a film basis. Paper or paper-like materials are also conceivable. Paper-like materials can be a thin layer of (disordered) fibers, which do not necessarily have to contain cellulose as the main component.

The antenna arrangement could be attached to the skin surface of the person by means of an adhesive film, that extends along the carrier structure, for example along the bottom side. The adhesive film can be designed as a hydrocolloid film, for example. If the dielectric constant of the adhesive film is between that of the carrier structure and that of the skin, a particularly good coupling of the high-frequency signal into the tissue can be achieved.

Different medical applications can be made possible by using such antenna arrangements. For example, the vocal tract of the person could be characterized or measured and an intended speech utterance could be determined on this basis. A corresponding system can include such an antenna arrangement, as well as an RF front end. The RF front end can be coupled to the one or more conductor structures in an electrically conductive manner (e.g., via a cable) and can be configured to perform radar measurements—for example, in transmission or reflection mode—based on the electromagnetic waves. A radar measurement in the sense of this disclosure can include, for example, the determination of the impulse response of the propagation channel. Thus, a radar measurement in the sense of this disclosure can include an interaction of electromagnetic waves with the tissue and the acquisition of information about this interaction. The RF front end could also be attached on the carrier structure. The RF front end can comprise, for example, a frequency mixer, an oscillator, an RF amplifier and a filter. It is conceivable that an extensively distributed battery mounted on the carrier structure is provided. In addition, the system may include a processor which is configured to determine the intended person's speech utterances on the basis of the radar measurements.

A corresponding method comprises the characterization of a person's vocal tract by using a radar measurement with a corresponding antenna arrangement.

For example, an intended speech utterance of the person could be determined based on the characterization of the vocal tract. It is conceivable to determine a text that describes a textual reproduction of the speech corresponding to the intended speech utterance. An audio output could be determined (i.e., an audio signal) that corresponds to the speech utterance. In the following, the term of the determination of a synthetic speech of a person is used for all such variants.

An antenna arrangement for electromagnetic waves in a near-body application is disclosed. For example, the antenna arrangement could be used to characterize a vocal tract. The vocal tract can be characterized, for example, to determine an intended speech utterance. The antenna arrangement has a top side and a bottom side. The antenna arrangement is designed to be applied with its bottom side to a person's skin surface. The antenna arrangement includes a two-dimensionally extended carrier structure. The carrier structure includes or consists of a textile. The antenna arrangement also includes one or more conductor structures. The conductor structures are designed, in coordination with the remaining parts of the arrangement, couple the electromagnetic waves into the tissue. Thus, they form an antenna.

By using a textile carrier structure, a particularly good contact can be established between the skin surface and the carrier structure. Furthermore, a textile carrier structure is comfortable to wear and can be worn for a longer period of time. This is particularly important for voice prostheses. The textile carrier structure can be breathable. The textile carrier structure can drain off or absorb sweat. Allergic reactions can be avoided.

The features and characteristics set forth above and those described below may be used not only in the corresponding explicitly explained combinations but also in further combinations or in isolation without departing from the scope of protection of the present invention.

### BRIEF DESCRIPTION OF THE DRAWINGS

FIG. 1 is a schematic lateral view of an antenna arrangement according to various examples.

FIG. 2 is a polar plot of the radiated electromagnetic power density provided by an antenna arrangement according to various exemplary embodiments with an asymmetrical radiation pattern.

FIG. 3 is a schematic top view of an antenna arrangement according to different exemplary embodiments.

FIG. 4 illustrates an exemplary scheme.

### DETAILED DESCRIPTION

The characteristics, features and advantages of the present invention described above, as well as the way in which they are achieved, will become clearer and more precisely understandable by means of the following description of the exemplary embodiments which will be discussed in more detail with reference to the figures.

In the following, the present invention will be discussed in more detail by means of preferred embodiments with reference to the figures. The same reference numerals in the figures denote the same or similar elements. The figures are schematic representations of various embodiments of the invention. The elements depicted in the figures are not necessarily shown to scale. Rather, the various elements illustrated in the figures are represented in such a way that their function and general purpose are understandable to those skilled in the art. Connections and couplings between functional entities and elements illustrated in the figures can also be implemented as indirect connections or couplings. A connection or coupling may be implemented in a wire-based or wireless fashion. Functional entities may be implemented as a hardware or software solution, or as a combined solution of hardware and software.

In the following, techniques are disclosed that allow to perform radar measurements in near-body applications. For example, medical applications could be made possible. Examples relate to the localization and/or tracking of surgical devices, such as an endoscope. Further examples relate to the detection of the position and/or movement of body structures, such as bones.

Another example relates to the characterization of a person's vocal tract. The characterization of the vocal tract makes it possible to determine a person's intended speech utterance. For example, individual sounds or a sequence of sounds could be determined; corresponding vowels or consonants or a corresponding sequence could be determined, for example. It is conceivable to determine a text that describes a textual reproduction of the speech. An audio output could be determined (i.e., an audio signal) that corresponds to the speech utterance. The term determination of a synthetic speech of a person is used hereinafter for all such variants.

In particular, synthesized speech can even be specified when the person is not able to form sounds. This means that “silent speech” is made possible. Voice disorders manifest themselves in a set of symptoms, which are grouped under the term dysphonia: from hoarseness to weak or distorted speech, to a complete loss of voice, known as aphonia. Voice disorders can have a functional or organic origin. Organic voice disorders can be further subdivided into structural or neurogenic disorders. A “silent speech” can be made possible for persons with such voice disorders.

Synthetic speech can also be generated if the person intentionally whispers or silently articulates. This could, for example, allow a private “conversation” over the phone.

In particular, antenna arrangements for electromagnetic waves that are suitable for near-body applications are disclosed. The corresponding antenna arrangements can be elastic or flexible and thin (e.g., not thicker than 5 mm or optionally not thicker than 2 mm) so that they can be stuck to the skin, for example. The antenna arrangements can have lateral dimensions that are substantially larger than the thickness (e.g., lateral dimensions of 2 to 5 cm and a thickness of 2 mm). The antenna arrangements can be particularly light and flexible so that they can be worn by the person even for a longer period of time.

The antenna arrangements also have advantageous radiation characteristics of electromagnetic waves. In particular, antenna arrangements are disclosed that preferentially emit electromagnetic waves into the person's tissue-instead of emitting them into the surrounding free space.

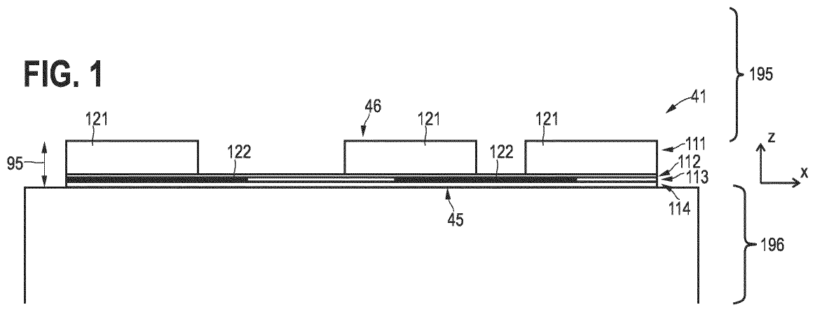

FIG. 1 is a schematic lateral view of an exemplary antenna arrangement 41. The antenna arrangement 41 is applied with its bottom side 45 to a skin surface 196 of the person. The antenna arrangement has a carrier structure 112 which is implemented as a carrier layer or carrier film here. A plurality of metallic conductor structures 122 (shown with black contrast in FIG. 1) are formed on the carrier structure 112. An appropriate metallic layer 113 is shown. They are configured to emit electromagnetic waves. An adhesive film 114 is provided underneath the metallic layer 113, but this is optional. The antenna arrangement 41 can be fixed to the skin surface 196 by means of the adhesive film 114 without an air gap or with a minimized one.

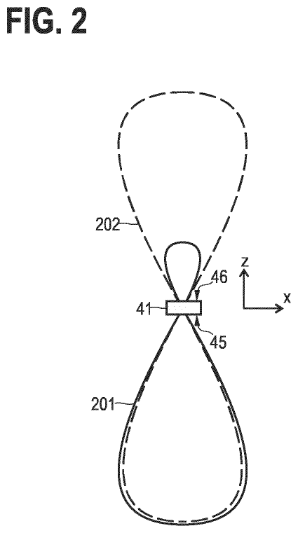

An absorbent material layer 111 facing the top side 46 of the antenna arrangement 41 is provided. In the illustrated example, this layer is formed by several absorber elements 121 (but it would also be possible to use only a single absorber element). In the example of FIG. 1, the absorbent material layer 111 is structured laterally (along the X-axis). The absorber elements 121 could be made of carbon, e.g., in the form of graphite, or metals such as chromium, titanium, iron or nickel, or a mixture of materials that can contain such materials. By using the absorbing material layer 111, a dielectric or magnetic loss mechanism is provided for the electromagnetic near field of the conductor structures 122. This leads to an asymmetrical radiation pattern of the electromagnetic waves, as shown in FIG. 2.

FIG. 2 illustrates aspects related to the radiation pattern. For the purpose of illustration, the antenna arrangement is assumed to be located in air or vacuum.

FIG. 2 illustrates aspects related to the radiation pattern. By way of illustration, the antenna arrangement is assumed to be located in air or vacuum.

FIG. 2 shows a section through the radiation pattern 201 (solid line) of the antenna arrangement 41 as a polar plot. It can be seen from FIG. 2 that the radiated power density adjacent to the bottom side (where the skin surface 196 is located in use) is greater than in the opposite free space adjacent to the top side 46. This means that the radiation pattern is asymmetrical. A relatively symmetrical radiation pattern 202 would result (dashed line) without the absorbing material layer 111.

In particular, the use of the absorber elements 121, which provide the dielectric, galvanic (resistive) or magnetic loss mechanism for the electromagnetic near field of the conductor structures 122, can ensure a compact antenna arrangement 41. The thickness 95 (see FIG. 1) of the antenna arrangement can be small relative to the longest wavelength of the emitted electromagnetic waves in vacuum, e.g., the thickness 95 could be not greater than 1/10 of this longest wavelength in vacuum.

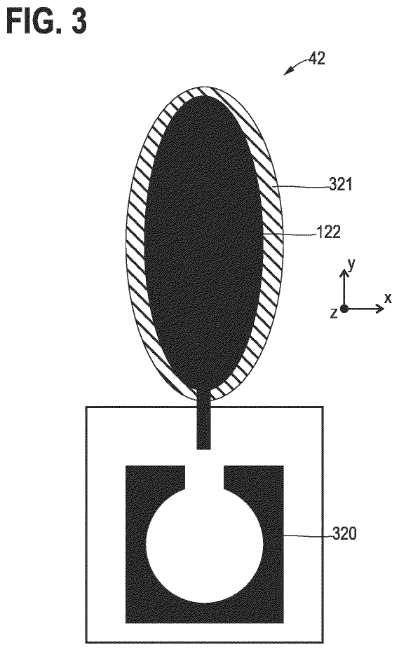

Such a thin implementation of the antenna arrangement 41 can also be achieved by employing other implementation variants of the absorber elements 121. A corresponding example is shown in FIG. 3.

FIG. 3 is a schematic top view of an exemplary antenna arrangement 42. The antenna arrangement 42 has also a metallic conductor structure 122 (which can be connected to a coaxial plug via contact pads 320; there is also a metallization on the underside of the contact pads 320, which is not shown in the top view of FIG. 3). The metallic conductor structure 122 is formed as a flat conductor plane; here it has an elliptical shape. A resistive layer 321 is arranged flush along a circumference of the flat conductor plane formed by the conductor structures 122. Thus, the resistive layer 321 acts as a resistance-producing element for the RF currents in the conductor structure 122. The resistive layer 321 could also be applied along the circumference of the conductor structure 122 and possibly beyond on the conductor structure 122, i.e., offset in the z-direction. As an alternative to the resistive layer 321, surface-mounted resistive elements could also be used, which are contacted, for example, via a further elliptical ring (outside the conductor structure 122). It is also conceivable that the resistive layer 321 extends vertically (in the z-direction) along an edge of the conductor structure 122.

Such an implementation of the antenna arrangement 42 can provide a resistive loss mechanism for electrical currents in the flat conductor plane. The current flow to the edge or circumference is reduced; and thus the radiation into the free space 195 is reduced (cf. FIG. 2).

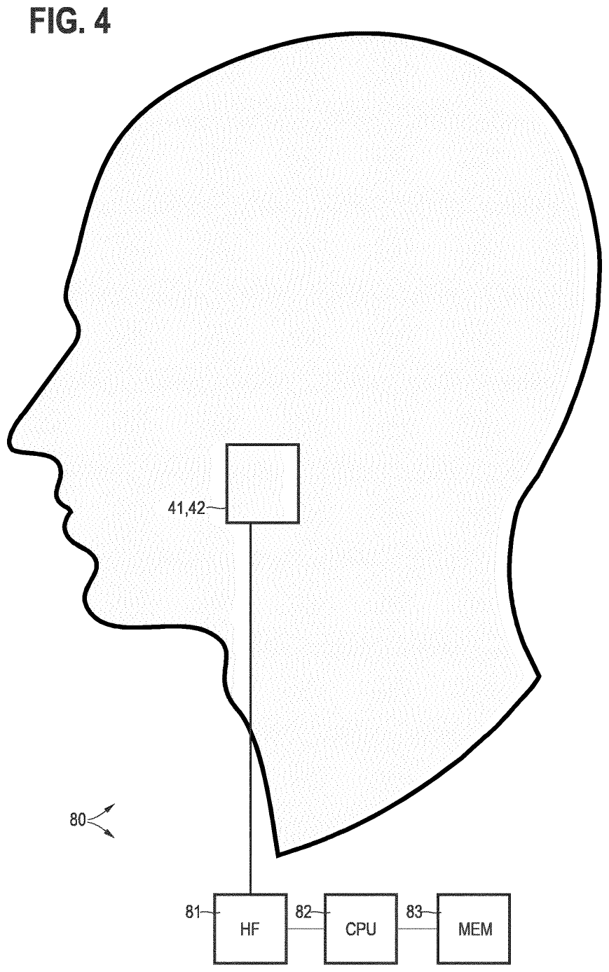

FIG. 4 illustrates an exemplary system 80. This system comprises the antenna arrangement 41 or the antenna arrangement 42 or a comparable antenna arrangement. In addition, an RF front end 81 and a processor (CPU) 82 are provided. This system is configured to carry out a radar measurement, for example, based on program code being loaded from the memory (MEM) 83. For this purpose, electromagnetic waves are transmitted into the person's tissue via the skin surface 196 (FIG. 1) by feeding a corresponding RF current flow into the conductor structures 122 (FIG. 2). Then, reflected or transmitted electromagnetic waves can be measured by means of a further antenna arrangement. Based on this radar measurement, the processor 82 could, for example, characterize the vocal tract, in order to determine an intended speech utterance.

To synthesize the speech, the digitized data received by the antenna can be processed and evaluated with regard to the signal-modulating changes in the vocal tract geometry on the free space route between the antennas. This evaluation can be carried out by using a machine-learned algorithm. The signal can be understood as the channel impulse response of the transmitted electromagnetic signal through the detected structures. It could then be evaluated using a machine-learned algorithm. In principle, corresponding techniques are known from: WO 2021/214065 so that further details do not need to be discussed here.

Of course, the features of the embodiments and aspects of the present invention described above can be combined with one another. In particular, the features can be used not only in the combinations described but also in other combinations or alone on their own without departing from the field of the invention.

For example, techniques for an antenna arrangement with a carrier structure, in particular with an elastic or flexible carrier structure, have been described above so that the antenna arrangement can be applied directly to a person's skin surface, for example without an air gap in between. The carrier structure can also be rigid, and in such a case the lateral dimensions should be small relative to the curvature of the parts of the face, e.g., smaller than 30 mm×30 mm.

It is conceivable to use the techniques, which are disclosed herein in connection with the use of one or more absorber elements that cause an asymmetrical radiation pattern of the electromagnetic waves, also for other types of antenna arrangements.

Examples include antenna arrangements to be integrated into glasses or a temple, into a mask or headphones, into a dental prosthesis or braces, into a dental implant, etc.

Furthermore, techniques have been described in which a textile carrier structure is used for an antenna arrangement. The textile carrier structure can be particularly helpful, if absorber elements are not used.

Furthermore, techniques have been described above in which antenna arrangements for electromagnetic waves are implemented. In various examples, it is not absolutely necessary to use antenna arrangements for electromagnetic waves in connection with radar measurements. It is conceivable, for example, to use the disclosed antenna arrangements to radiate RF energy into the body in order to increase the temperature in the tissue. A data transmission could also be implemented, for example from or to implants.

1. An antenna arrangement for electromagnetic waves in a near-body application, wherein the antenna arrangement is designed to be attached with its bottom side onto a skin surface of a person, wherein the antenna arrangement comprises:
a two-dimensionally extended carrier structure
one or more conductor structures that are attached to the carrier structure and that are configured to emit electromagnetic waves, and
one or more absorber elements which are attached to the carrier structure and which are configured to cause a radiation pattern of the electromagnetic waves that is asymmetrical with respect to the bottom side and a top side of the antenna arrangement opposite the bottom side.

2. The antenna arrangement according to claim 1,
wherein the one or more absorber elements provide a resistive loss mechanism for electric currents in the one or more conductor structures.

3. The antenna arrangement according to claim 1,
wherein the one or more conductor structures form a flat conductor plane,
wherein the one or more absorber elements comprise one or more resistors which are arranged on a circumference of the flat conductor plane and are connected thereto.

4. The antenna arrangement according to claim 3,
wherein the one or more resistors are attached onto the flat conductor plane and project beyond the circumference of the flat conductor plane.

5. The antenna arrangement according to claim 4,
wherein the one or more resistors have an extension perpendicular to the flat conductor plane.

6. The antenna arrangement according to claim 1,
wherein the one or more absorber elements provide a dielectric or magnetic loss mechanism for an electromagnetic near field of the one or more conductor structures

7. The antenna arrangement according to claim 1,
wherein the one or more absorber elements form one or more two-dimensionally extended absorbent material layers.

8. The antenna arrangement according to claim 7,
wherein absorbent material of the one or more absorbent material layers is selected from the group consisting of: carbon; metal; carbon particles in a carrier material; metal particles in a carrier material.

9. The antenna arrangement according to claim 7,
wherein the one or more absorbent material layers have a lateral structuring.

10. The antenna arrangement according to claim 9,
wherein the lateral structuring of the one or more absorbent material layers correlates with a lateral structuring of the one or more conductor structures.

11. The antenna arrangement according to claim 1,
wherein a thickness of the antenna arrangement is not greater than 1/10 of a wavelength of the longest wavelength component of the electromagnetic waves in vacuum.

12. The antenna arrangement according to claim 1,
wherein the carrier structure is designed as a textile layer.

13. The antenna arrangement according to claim 12,
wherein the one or more conductor structures are fixed as threads or ribbons to the textile layer.

14. The antenna arrangement according to claim 12,
wherein the one or more conductor structures are embroidered onto the textile layer.

15. The antenna arrangement according to claim 1,
wherein the carrier structure and/or the one or more conductor structures are made of film or paper or paper-like material.

16. The antenna arrangement according to claim 1, further comprising:
an adhesive film which extends along the bottom side

17. A system comprising:
the antenna arrangement according to claim 1,
a radio frequency front end that is coupled to the one or more conductor structures and is configured to perform radar measurements based on the propagation of electromagnetic waves, and
a processor configured to characterize, based on the radar measurements, the person's vocal tract to determine an intended speech utterance of the person.

18. A method comprising:
characterizing a person's vocal tract by means of a radar measurement using the antenna arrangement according to claim 1, and
determining an intended speech utterance of the person based on the characterization.
